(19) United States
(12) Patent Application Publication
(10) Pub. No.: US 2015/0201970 A1
(43) Pub. Date: Jul. 23, 2015

(54) DYNAMIC SPINAL STABILIZATION ROD

(71) Applicant: Joshua AFERZON, (US)

(72) Inventor: Joshua Aferzon, Stamford, CT (US)

(21) Appl. No.: 14/414,063

(22) PCT Filed: Jul. 11, 2013
(86) PCT No.: PCT/US2013/050164

§ 371 Date: Jan. 9, 2015

Related U.S. Application Data
(60) Provisional application No. 61/670,193, filed on Jul. 11, 2012.

(51) Int. Cl. A61B 17/70

(52) U.S. Cl. CPC A61B 17/7031; A61B 17/7034

(57) ABSTRACT

A dynamic spinal stabilization rod is disclosed including an elongate core, a first plurality of rigid components and a second plurality of flexible components. Each of the first plurality of rigid components may include a first central aperture. The elongate core may be configured to extend through the first central aperture of each rigid component. Also, the first plurality of rigid components may each include a locking mechanism for fixedly securing the rigid component to the elongate core. Each of the flexible components may include a second central aperture. Also, the elongate core may be configured to extend through the second central aperture of each of the flexible components. The first plurality of rigid components, the second plurality of flexible components and at least a portion of the elongate core may be configured to be disposed between two immediately adjacent ones of the at least two bone anchors.

### RELATED APPLICATIONS

The present non-provisional patent application claims the benefit of priority to U.S. Provisional Patent Application No. 61/670,193 filed Jul. 11, 2012 to the same inventor, the entire contents of which are hereby incorporated by reference.

### BACKGROUND

The present disclosure relates to surgical implants for orthopedic surgery, namely spinal stabilizing rods.

Pedicle screw systems used in spinal stabilization applications for posterior fixation of the vertebral column typically utilize rods to stabilize bone screws between vertebrae. Contemporary vertebral rods are rigid constructs designed to bridge vertebrae and maintain relative positioning of the pedicle screw and the vertebrae to which they are anchored. However, such rigid vertebral rod assemblies limit natural articulation and associated motion of the spine. This causes a great risk of implant failure for younger and more active patients who may stress static implants during normal movement. Also, even without implant failure, the stress on static implants may cause adjacent-level damage to the spine and surrounding tissue.

### SUMMARY

In an embodiment, a dynamic spinal stabilization rod for connecting at least two bone anchors in a spine of a patient is disclosed. The dynamic spinal stabilization rod may include an elongate core, a first plurality of rigid components and a second plurality of flexible components. The elongate core may be configured to resist tension along its own longitudinal extent. Each of the first plurality of rigid components may include a first central aperture. The elongate core may be configured to extend through the first central aperture of each of the rigid components. Also, at least two of the first plurality of rigid components may each include a locking mechanism for fixedly securing the rigid component to the elongate core. Each of the flexible components may include a second central aperture. Also, the elongate core may be configured to extend through the second central aperture of each of the flexible components. Additionally, the first plurality of rigid components may be substantially harder than the second plurality of flexible components. Further, the first plurality of rigid components, the second plurality of flexible components and at least a portion of the elongate core may be configured to be disposed between two immediately adjacent ones of the at least two bone anchors.

In one or more embodiments, the first plurality of rigid components and the second plurality of flexible components may be arranged in an alternating pattern along the longitudinal extent of the elongate core. The first plurality of rigid components and the second plurality of flexible components may be arranged in a symmetrical pattern along the longitudinal extend of the elongate core. Also, each of the first plurality of rigid components may include a locking mechanism for fixedly securing a respective one of the first plurality of rigid components to the elongate core. The locking mechanism may include a gap in the respective one of the at least two rigid components, the gap extending between two opposed ends of the respective one of the at least two rigid components and extending from the first central aperture to an outer perimeter of the respective one of the at least two rigid components. The two opposed ends may be biased toward closing the gap for tightly gripping the elongate core by constricting the respective one of the at least two rigid components around the elongate core. The locking mechanism may include a screw extending through the respective one of the at least two rigid components, wherein the screw grips the elongate core. Further, the locking mechanism may include the first central aperture tightly gripping the elongate core. Further still, the locking mechanism may include a friction hold from side walls of the first central aperture frictionally engaging the elongate core.

Additionally in one or more embodiments a bone screw forming each of the at least two bone anchors may be provided. The bone screw may be configured to anchor into the spine. Also, the bone screw may include a screw head and a head nut, wherein the screw head includes a cradle for receiving more than one of the first plurality of rigid components therein. The head nut may be threadedly engaged with the screw head and secures the more than one of the first plurality of rigid components in the cradle.

Further embodiments may include various means for performing functions corresponding to the elements and methods discussed herein.

### BRIEF DESCRIPTION OF DRAWINGS

The accompanying drawings are presented to aid in the description of embodiments of the disclosure and are provided solely for illustration of the embodiments and not limitation thereof.

FIG. 1 illustrates a perspective view of a dynamic spinal stabilization rod in accordance with an embodiment.

FIG. 2 illustrates a top view of the dynamic spinal stabilization rod of FIG. 1.

FIG. 3 illustrates a perspective view of a rigid component with locking screw in accordance with an embodiment.

FIG. 4 illustrates a perspective view of a flexible component in accordance with an embodiment.

FIG. 5 illustrates a side elevation view of a spine of a patient with a dynamic spinal stabilization rod in partial perspective view aligned for anchoring to the spine in accordance with an embodiment.

FIG. 6 illustrates a perspective view of a dynamic spinal stabilization rod without the bone screws in accordance with an embodiment.

FIG. 7 illustrates a perspective partially exploded view of an alternative dynamic spinal stabilization rod without the bone screws in accordance with an embodiment.

FIG. 8 illustrates a perspective view of an alternative rigid component in accordance with an embodiment.

FIG. 9. illustrates a perspective view of an elongate core in accordance with an embodiment.

FIG. 10 illustrates a perspective view of a further alternative rigid component in accordance with an embodiment.

FIG. 11 illustrates an exploded perspective view of the rigid component of FIG. 10.

FIG. 12 is a process flow of a method of forming a dynamic spinal stabilization rod in accordance with an embodiment.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the disclosure or the claims. Alternate embodiments may be devised without departing from the scope of the disclosure. Additionally, well-known elements of the disclosure will not be described in detail or will be omitted so as not to obscure the relevant details of the disclosure.

The word “exemplary” is used herein to mean “serving as an example, instance, or illustration.” Any implementation described herein as “exemplary” is not necessarily to be construed as preferred or advantageous over other implementations. Additionally, use of the words “first” and “second” or similar verbiage is intended herein for clarity purposes to distinguish various described elements and is not intended to limit the invention to a particular order or hierarchy of elements. Further, the word “plurality” is intended herein to refer to more than one element. A plurality may include as few as two elements, a large number of elements or any number in between.

The word “mechanism” as used herein refers to a thing made or adapted for a particular purpose, especially a device with discrete parts and/or integral elements that work together. A mechanism may include a single unitary part or a plurality of parts. Additionally, the terms “mechanism,” “device” and “apparatus” are used herein interchangeably.

The word “aperture” as used herein refers to an opening, hole or gap passing entirely through a material or portion of a structure. Also, the word “gap” as used herein refers to an elongate narrow aperture or slit in a portion of a material element. An aperture or gap need not have a uniform, symmetric or simple geometric cross-sectional shape.

In an embodiment a dynamic spinal stabilization rod is disclosed that utilizes an elongate core threaded through alternating rigid and flexible components. The rigid components may be locked onto the elongate core, thereby fixing their position along the length of the elongate core, as well as holding the flexible components in position. The rigid components should be substantially stiffer than the flexible components. The flexible components are designed to offer flexion, compression, elongation, and other motions preserving the dynamic nature of their material properties. The flexible components should be substantially more elastic and bendable than the rigid components. The elongate core may maintain the coordinated pattern of the rod, as well as resist tensile forces longitudinally between rigid components. In an embodiment, the rigid components and flexible components are beaded onto the elongate core in an alternating pattern, where the rigid components may be tightly secured to the elongate core to provide rigid focal points.

In accordance with the various embodiments, the disclosed dynamic spinal stabilization rod enables pedicle screw integration at choice locations along the longitudinal extent of the rod. No longer is the range of usable rod lengths limited, forcing surgeons to use lengths of rod not perfectly suited to the patient. The disclosed embodiments enable surgeons to trim the elongate core to a desired length, while still having sufficient focal points with which to secure the custom length rod to a pedicle screw spacing dictated by a patient's anatomy. The distance between the rigid components and flexible components may be calculated such that at least one rigid component will fall within the head of the pedicle screw during surgical installation. This reliably allows the rod to be fixated to the pedicle screws at the focal points. Also, current systems include bulky components with complicate delivery schemes, increased surface area contact, which may be hard on immunosensitive tissues. The disclosed technologies provide a smaller profile, which allows the rod to be secured with a percutaneous minimally invasive technique.

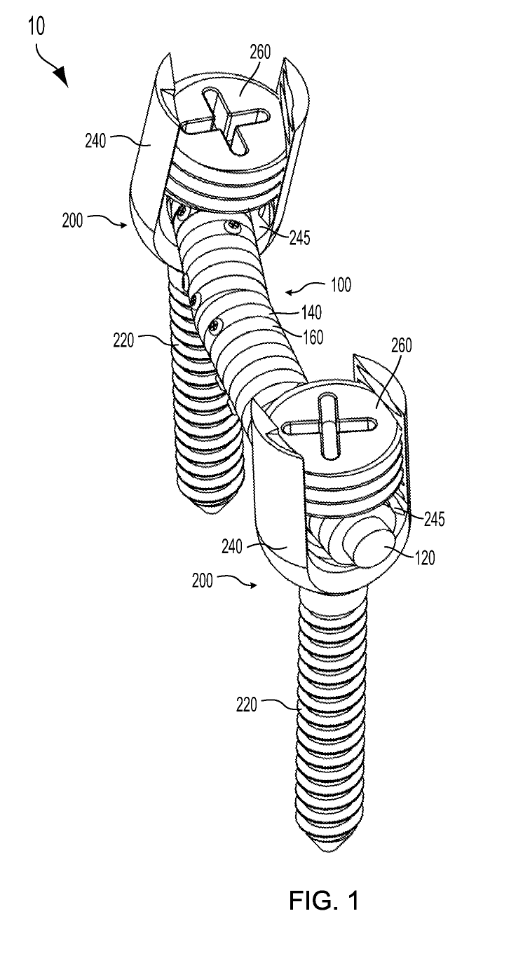

FIG. 1 illustrates a perspective view of an exemplary dynamic spinal stabilization rod 10, including two bone anchors 200 at opposed ends of a central rod assembly 100. The central rod assembly 100 includes an elongate core 120, several rigid components 140 and several flexible components 160. The elongate core 120 may be configured to resist tension along a longitudinal extent of the central rod assembly 100 and the elongate core itself. The plurality of rigid components (referred to as the “first plurality of rigid components”) include individual rigid components 140 each of which include a central aperture (referred to as the “first central aperture”). The elongate core 120 extends through the first central aperture of each of the rigid components. The plurality of flexible components (referred to as the “second plurality of flexible components) includes individual flexible components 160 each of which include their own central aperture (referred to as a “second central aperture” as distinguished from the first central aperture of the rigid component). The elongate core 120 extends through the second central aperture of each of the flexible components. Together, the first plurality of rigid components, the second plurality of flexible components and the elongate core are configured to be disposed between two immediately adjacent bone anchors 200.

The dynamic spinal stabilization rod 10 may include bone anchors 200 in the form of bone screws. The bone anchors 200 may be configured to anchor into the spine of a patient, particularly using the lower portion 220 formed as a threaded screw. An upper end of the bone anchor 200 may include a screw head 240 and a head nut 260. The screw head 240 includes a cradle 245 for receiving therein one end of the central rod assembly 100. The head nut 260 threadedly engages the screw head 240 and secures at least a couple of rigid components 140 in the cradle 245. Threadedly engaging refers to a helical ridge on the outside of one element being mated with a matching helical ridge on the inside of another element so that the two elements may be screwed together. The screw head 240 may be a polyaxial mechanisms configured to articulate (pivot and twist) relative the lower portion 220.

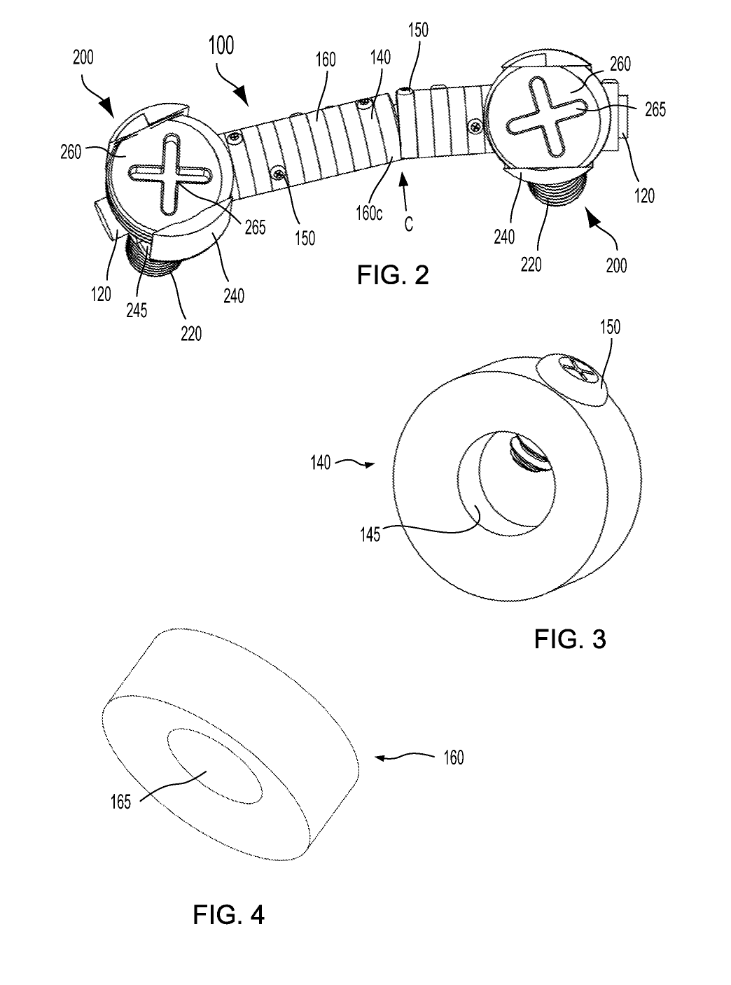

FIG. 2 illustrates a top view of the dynamic spinal stabilization rod 10 of FIG. 1. In this embodiment all of the rigid components 140 include a locking mechanism 150 (illustrated as a fixing screw) for fixedly securing each rigid component 140 to the elongate core 120, however not all rigid components need be fixedly secured to the elongate core. Preferably, at least the rigid components 140 held under the screw head 260 inside the cradle 245 include a locking mechanism. As used herein, the expression “fixedly secured” or “fixedly securing” refers to securely attaching or fastening elements together so they do not easily or readily come apart. Each of the rigid components 140 fixedly secured by being sandwiched inside the cradle 245 are considered a rigid focal point. The rigid focal points transfer loads between the central rod assembly 100 and the bone anchors 200 and vise-versa. Although not shown in the drawings, the dynamic spinal stabilization rod may extend across more than two bone anchors. For example, each of three consecutive vertebrae may respectively have a pedicle screw secured thereto and a single central rod assembly 100 may be installed across and secured to all three pedicle screws.

As shown in FIGS. 1 and 2, the screw head 240 may include threaded inner walls that form the vertical walls of the cradle 245. The threaded inner walls are configured to mate with the threads of the head nut 260. The head nut 260 may be screwed into the cradle 245 using the top bore 265 (shown as a Phillips-type screw head). In this way, once a portion of the central rod assembly 100 is seated in the cradle 245, with that portion of the central rod assembly 100 being sandwiched between the head nut 260 and the cradle 245. This configuration also enables the broad lower surface of the head nut 260 to extend across and secure multiple rigid components 140 and possibly multiple flexible components 160. An alternative screw head design may include threading on the outside of the vertical walls with the head nut formed as a more traditional nut with a threaded central hole sized to wrap around and threadedly engage the threading on the outside of the alternative screw head vertical walls.

It should be noted that FIG. 2 also illustrates the dynamic aspect of dynamic spinal stabilization rod 10. A central portion (noted as “C”) of the central rod assembly 100 is bent, compressing at least one flexible component 160c, which is sandwiched on two sides by rigid components 140 that are far less yielding.

FIG. 3 illustrates a perspective view of an exemplary rigid component 140 by itself. The rigid component 140 may be composed of an implantable metal, such as titanium alloy. Alternatively, the rigid component 140 may be made of another sturdy material such as a polymer, ceramic, a composite of those materials or the like. While other materials and combinations of materials are possible, the rigid components should be stiffer than the flexible components. As used herein, the terms “rigid” and “stiff” refer to a materials inability to or tendency not to bend, change or be forced out of shape. The body of the rigid component 140 includes a central aperture 145 extending there through. An outer perimeter of the rigid component may be configured to integrate with a pedicle screw head during fastening of the rod. The first central aperture 145 may be adapted to provide an opening through which an elongate core may threaded.

The rigid component 140 also includes some form of locking mechanism. In the embodiment shown in FIG. 3, the locking mechanism includes a fixing screw 150 extending through the rigid component 140 for gripping and thereby locking onto the elongate core. Gripping refers to maintaining a firm contact between two elements as to create a high enough level of friction between those two elements as to prevent or resist relative movement thereof. Similarly, a “friction hold” maintains a high enough level of friction to prevent or resist relative movement. Once the rigid component 140 is mounted on the elongate core and the fixing screw installed as shown, the fixing screw 150 may bite into the elongate core in order to lock the rigid component in place. By locking onto the elongate core, the screw 150 may prevent displacement or rotation of the rigid component 140 relative to at least a segment of the elongate core. Other locking mechanism techniques may be used, some of which are disclosed herein.

FIG. 4 illustrates a perspective view of an exemplary flexible component 160 by itself. The flexible component 160 may be made from a flexible material such as a polymer or composite of materials. While various materials and combinations of materials are possible, the flexible components should be more elastic and bendable than the rigid components. As used herein, the term “flexible” refers to the opposite of rigid and thus a materials ability to easily bend, change or be forced out of shape. A resilient material will have a tendency to recoil or spring back into shape after bending, stretching or being compressed. The flexible component may be resilient as well as flexible. For example, the flexible component 160 may be formed from Polyether ether ketone (PEEK), Polytetrafluoroethylene (PTFE), Poly(methyl methacrylate) (PMMA), Polyethylene or Silicone. In this way, when the spine of an active individual demands some bending, twisting or compression from the central rod assembly, the flexible components will accommodate such demands to a limited extent. The flexible component 160 also includes its own central aperture 165 (referred to as a “second central aperture” as distinguished from the first central aperture of the rigid component) extending there through. The second central aperture 165 may be adapted to provide an opening through which the elongate core may be threaded.

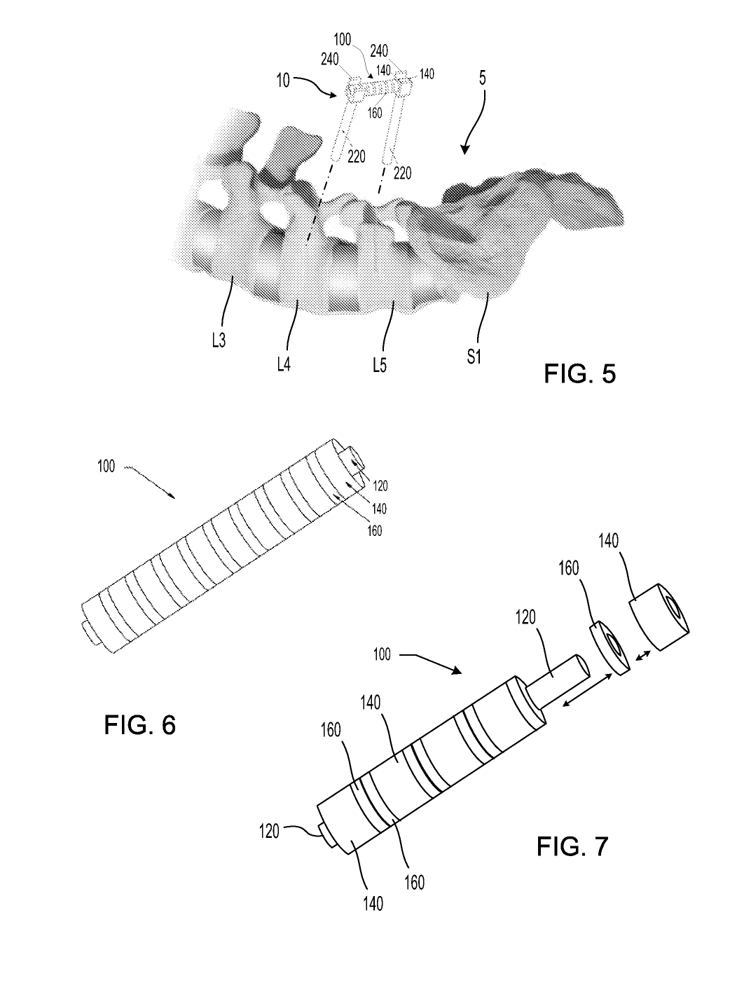

FIG. 5 illustrates a side elevation view of the lumbar portion of a human spine 5. Three lower lumbar vertebrae L3, L4, L5 and the sacrum S1 are shown with a dynamic spinal stabilization rod 10 being aligned for anchoring to the spine 5. The lower portion 220 of each bone anchor (shown as simplified representations of pedicle screws) may be secured in a hole drilled through the boney portions of the spine. This may be done prior to installing the central rod assembly 100 between the two bone anchors. In this illustration, the head nuts are not included in order to more clearly show how multiple rigid components 140 and even multiple flexible components 160 may be fit in the cradle of each screw head 240. Preferably, the distances between the rigid components 140 along the elongate core are such that at least one rigid component 140 is able to integrate with the screw head 240. This should structurally anchor the entire system. The flexible components 160 further provide motion stabilization as the bone anchors begin to migrate under normal forces of movement.

FIG. 6 illustrates a perspective view of the central rod assembly 100 of FIG. 5. The central rod assembly 100 includes alternating rigid components 140 and flexible components 160 threaded onto an elongate core 120 like beads on a wire. This configuration provides a combination of support and flexibility throughout the central rod assembly 100. Other coordinated patterns and variations of the rigid and flexible components may be possible. For example, FIG. 7 illustrates a perspective partially exploded view of an alternative central rod assembly 100. As shown, one rigid component 140 and one flexible component 160 have been removed from (or have not yet been installed on) the elongate core 120. In this embodiment, two flexible components 160 are stacked side-by-side, followed by a single rigid component 140. The pattern repeats but the central rod assembly 100 should include at least one rigid component 140 fixedly secured to the ends of the elongate core 120.

The elongate core 120 may be composed of a flexible high tensile strength material such as polymer that can be threaded through a rigid component 140 and flexible component 160. Other materials and combinations of materials are possible. The elongate core 120 may be further adapted to maintain tension throughout the system and stabilize overall rod motion.

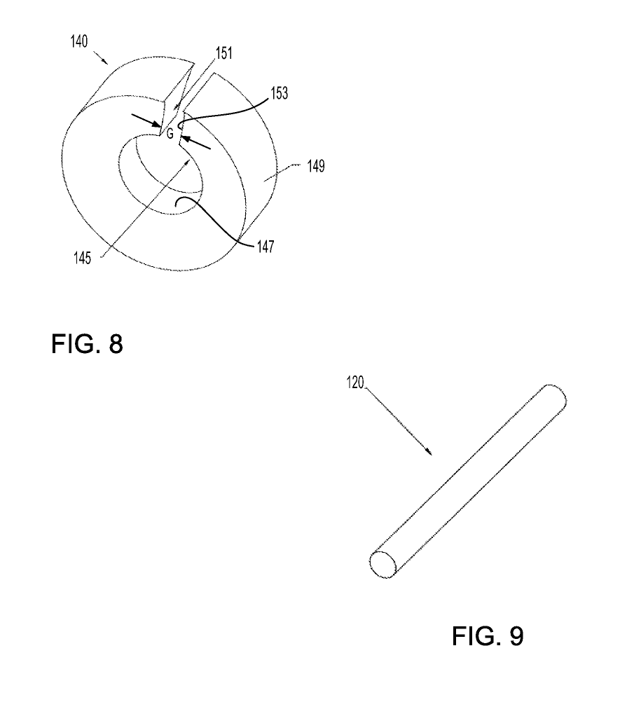

FIG. 8 illustrates a perspective view of an alternative rigid component in accordance with an embodiment. In this embodiment, the locking mechanism includes a gap G in the rigid component in which it is formed. The gap extends between two opposed ends 151, 153 of the rigid component and extends from an inner wall 147 of the first central aperture 145 to an outer perimeter 149 of the rigid component. The rigid component 140 may be formed so that before being mounted on the elongate core, the two opposed ends 151, 153 either touch or almost touch. Thus, the two opposed ends 151, 153 may be pulled away from one another, causing the gap G to increase in size. This results in the two opposed ends 151, 153 being biased toward closing the gap for tightly gripping the elongate core. The rigid component, while being rigid may also be sufficiently resilient as to have a tendency to constrict around and thereby grip the elongate core.

FIG. 9. illustrates a perspective view of an elongate core in accordance with the various embodiments. The elongate core 120 may be significantly longer along its longitudinal extent as compared to its width. By itself, the elongate core 120 may be formed like a rod. The elongate core 120 may be made from a flexible high tensile strength material such as a polymer, metal or a composite of those materials or the like. While other materials and combinations of materials are possible, the elongate core should be stiffer than the flexible components, as well as more elastic and bendable than the rigid components. For example, the elongate core may be made from Polyethylene or almost any suitable material that may be made into a thread.

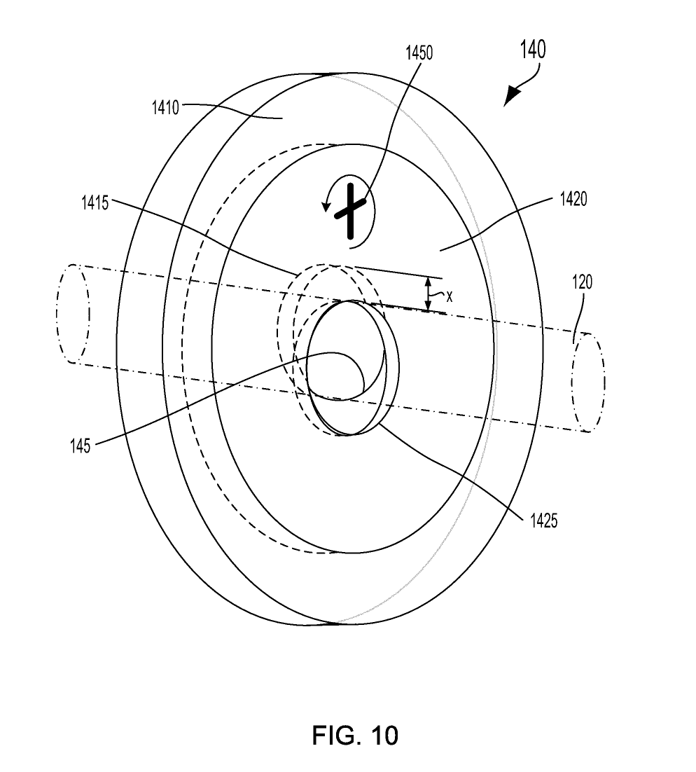

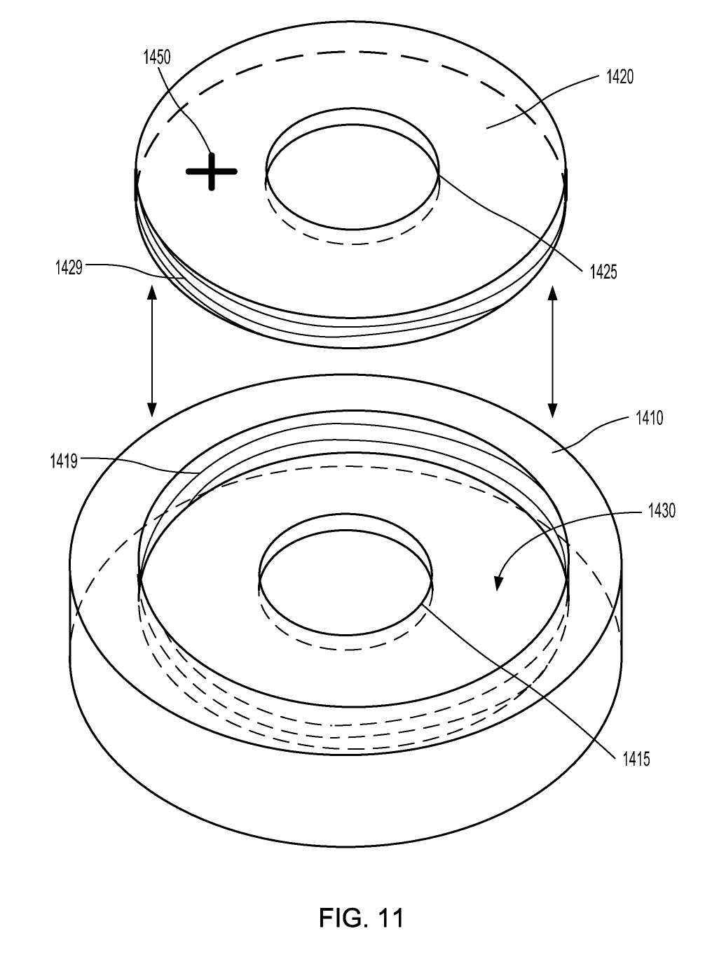

FIG. 10 illustrates a perspective view of a further alternative rigid component 140 in accordance with an embodiment. The rigid component 140 may be formed from two discrete segments 1410, 1420. FIG. 11 illustrates an exploded perspective view of the same embodiment rigid component shown in FIG. 10, which more clearly shows the two discrete segments 1410, 1420 individually. An outer segment 1410 receives the inner segment 1420 in a seat 1430. The seat 1430 is formed as a depression in the outer segment 1410 sized to precisely receive the inner segment 1420. Both of the two discrete segments 1410, 1420 may include male/female threading 1419, 1429, respectively, in order to fixedly secure the combined structure to form a whole rigid component 140. Also, while the two discrete segments 1410, 1420 may be formed of the same material, they may alternatively be formed of different materials. For example, the inner segment 1410 may be more or less rigid than the outer segment 1420.

The outer segment 1410 includes a first offset aperture 1415 and the inner segment 1420 includes a second offset aperture 1425. At least one of these apertures 1415, 1425 is formed slightly off center. In this way, these apertures 1415, 1425 are configured to be offset or misaligned from one another once the inner segment 1420 is installed in the seat 1430 of the outer segment 1410 (as shown in FIG. 10). Also, one or both of the apertures 1415, 1425 should be larger than an outer diameter of the elongate core. As with previous embodiments, when the two discrete portions 1410, 1420 are joined, the rigid component 140 still forms a generally annular or cylindrical shape, having a first central aperture 145. The offset x between the first offset aperture 1415 and the second offset aperture 1425 causes the two discrete segments 1410, 1420 to pinch and thus grip the elongate core 120 (shown in phantom in FIG. 10) extending through the rigid component 140.

Thus, these rigid components include this alternative locking mechanism, which may be formed by an outer segment 1410 and an inner segment 1420. The outer segment 1410 and the inner segment 1420 are discrete from one another, but may be fixedly secured to one another in a mated configuration to form an single integral rigid component 140. The outer segment 1410 includes a cylindrical recess formed as a seat 1430 for matingly receiving the inner segment 1420. An inner diameter of the cylindrical recess may be sized to match an outer diameter of the inner segment 1420. Also, a cylindrical wall of the cylindrical recess 1430 may include a first threading configured to receive and mate with an outer perimeter of the inner segment 1420 that includes a second threading. In this way, the inner segment 1420 screws into the cylindrical recess 1430 of the outer segment to fixedly secure the inner and outer segments together. Additionally, the outer segment 140 includes a first offset aperture 1415 and the inner segment 1420 includes a second offset aperture 1425. The first and second offset apertures 1415, 1425 are offset x from one another and at least one of the offset apertures 1415, 1425 is not centered in the respective inner/outer segment in which it is formed. An outer diameter of the elongate core may be substantially smaller than an inner diameter of at least one of the offset apertures.

To assemble the two discrete segments 1410, 1420 on the elongate core 120, the outer segment 1410 may be threaded onto the elongate core roughly into the desired position along the longitudinal length of the elongate core. Then the inner segment 1420 may be threaded onto the elongate core until it joins with the outer segment 1410. Holding the outer segment 1410 and the elongate core 120 in a fixed position relative to one another, the inner segment 1420 may then be screwed into the seat 1430. A small Phillips type screw recess 1450 may be provided on a planar surface of the inner segment 1420 in order to facilitate screwing the inner segment 1420 into the outer segment 1410. Although the screw recess 1450 is disposed offset from a center of the inner segment 1420, a clockwise/counter-clockwise rotation should still work to screw the overall inner segment 1420 into the outer segment 1410. As the inner segment 1420 approaches a fully seated position within the seat 1430, opposed walls of the offset apertures 1415, 1425 should pinch together to grip the elongate core 120, thus providing the locking mechanism.

In various embodiments, the dimensions of the dynamic spinal stabilization rod 10 or the individual elements may be modified as desired. The particular proportional sizes shown in the accompanying drawings are for illustrative purposes.

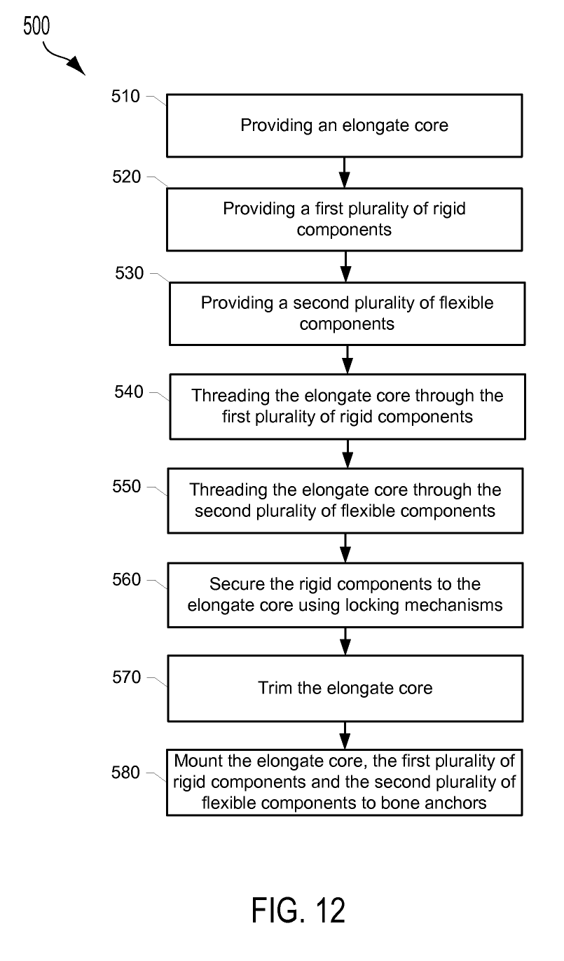

FIG. 12 illustrates a process flow for an embodiment method 500 of using a dynamic spinal stabilization rod. In block 510 an elongate core may be provided that is configured to resist tension along its longitudinal extent. In block 520 a first plurality of rigid components may be provided. The rigid components may be any of the variety of rigid components described above. In block 530 a second plurality of flexible components may be provided. The flexible components may be any of the flexible components described above. In block 540, the elongate core may be threaded through the first central recess of one of the first plurality of rigid components. In block 550, the elongate core may be further threaded through the second central recess of one of the second plurality of flexible components. Blocks 540 and 550 may be alternated in order to mount the rigid components and flexible components on the elongate member in a desirable pattern. In doing so, a flexible component is made to abut and is flanked on opposed sides by two rigid components. Also, it may be desirable to ensure that a rigid component is disposed on the two opposed ends of the series of rigid and flexible components. In this way, the first plurality is greater than the second plurality. In block 560 the rigid components may be fixedly secured to the elongate core using the locking mechanism. Securing the rigid components to the elongate core may be done as each individual rigid component is mounted on the elongate core or after all or most of the rigid and flexible components are mounted on the elongate core. In block 570, a length of the elongate core may be trimmed once a desired length is achieved for dynamic spinal stabilization rod. Alternatively, the length may be trimmed before all or any the rigid and flexible components are mounted onto the elongate rod. Once a desired number of rigid components and flexible components are mounted on the elongate core and the elongate core is of a desirable length, this portion of the assembly may be referred to as a central rod assembly. In block 580, the central rod assembly may be mounted to at least two bone anchors. The bone anchors may be pedicle screws already installed in adjacent vertebrae of the spine of a patient. Mounting the central rod assembly to at least two bone anchors may fixedly secure at least one rigid component to each of the two bone anchors. In fact, mounting the central rod assembly to at least two bone anchors may fixedly secure at least two rigid components to each of the two bone anchors.

The foregoing method descriptions and the process flow diagrams are provided merely as illustrative examples and are not intended to require or imply that the blocks of the various embodiments must be performed in the order presented. As will be appreciated by one of skill in the art the order of blocks in the foregoing embodiments may be performed in any order. Also, the various illustrative logical blocks and process flow diagram blocks described in connection with the embodiments disclosed herein may be implemented as an apparatus manipulated by human hand. Skilled artisans may implement the functionality described herein in varying ways for each particular application, but such implementation decisions should not be interpreted as causing a departure from the scope of the present invention.

Words such as “thereafter,” “then,” “next,” etc. are not intended to limit the order of the blocks; these words are simply used to guide the reader through the description of the methods. Further, any reference to claim elements in the singular, for example, using the articles “a,” “an” or “the” is not to be construed as limiting the element to the singular.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

What is claimed is:

1. A dynamic spinal stabilization rod for connecting at least two bone anchors in a spine of a patient, comprising:
an elongate core configured to resist tension along a longitudinal extent thereof;
a first plurality of rigid components, wherein each of the first plurality of rigid components includes a first central aperture, the elongate core being configured to extend through the first central aperture of each of the first plurality of rigid components, wherein at least two of the first plurality of rigid components each include a locking mechanism for fixedly securing a respective one of the at least two of the first plurality of rigid components to the elongate core;
a second plurality of flexible components, wherein each of the second plurality of flexible components includes a second central aperture, the elongate core being configured to extend through the second central aperture of each of the second plurality of flexible components, the first plurality of rigid components being stiffer than the second plurality of flexible components, wherein the first plurality of rigid components, the second plurality of flexible components and at least a portion of the elongate core are configured to be disposed between two immediately adjacent ones of the at least two bone anchors.

2. The dynamic spinal stabilization rod of claim 1, wherein the first plurality of rigid components and the second plurality of flexible components are arranged in an alternating pattern along the longitudinal extend of the elongate core.

3. The dynamic spinal stabilization rod of claim 1, wherein the first plurality of rigid components and the second plurality of flexible components are arranged in a symmetrical pattern along the longitudinal extend of the elongate core.

4. The dynamic spinal stabilization rod of claim 1, wherein each of the first plurality of rigid components includes the locking mechanism for fixedly securing a respective one of the first plurality of rigid components to the elongate core.

5. The dynamic spinal stabilization rod of claim 1, wherein the locking mechanism includes a screw extending through the respective one of the at least two of the first plurality of rigid components, wherein the elongate core is gripped by the screw.

6. The dynamic spinal stabilization rod of claim 1, wherein the locking mechanism includes a gap in the respective one of the at least two of the first plurality of rigid components, the gap extending between two opposed ends of the respective one of the at least two of the first plurality of rigid components and extending from the first central aperture to an outer perimeter of the respective one of the at least two of the first plurality of rigid components, wherein the two opposed ends are biased toward closing the gap for tightly gripping the elongate core by constricting the respective one of the at least two of the first plurality of rigid components around the elongate core.

7. The dynamic spinal stabilization rod of claim 1, wherein the locking mechanism includes the first central aperture tightly gripping the elongate core.

8. The dynamic spinal stabilization rod of claim 1, wherein the locking mechanism includes a friction hold from side walls of the first central aperture frictionally engaging the elongate core.

9. The dynamic spinal stabilization rod of claim 1, wherein the first plurality of rigid components is greater in number than the second plurality of flexible components.

10. The dynamic spinal stabilization rod of claim 1, further comprising:
a bone screw forming each of the at least two bone anchors, the bone screw configured to anchor into the spine, the bone screw including a screw head and a head nut, wherein the screw head includes a cradle for receiving more than one of the first plurality of rigid components therein, wherein the head nut threadedly engages the screw head and secures the more than one of the first plurality of rigid components in the cradle.

11. A method of forming a dynamic spinal stabilization rod, the method comprising:
a) providing an elongate core configured to resist tension along a longitudinal extent thereof;
b) providing a first plurality of rigid components, wherein each of the first plurality of rigid components includes a first central aperture, the elongate core being configured to extend through the first central aperture of each of the first plurality of rigid components, wherein at least two of the first plurality of rigid components each include a locking mechanism for fixedly securing a respective one of the at least two of the first plurality of rigid components to the elongate core;
c) providing a second plurality of flexible components, wherein each of the second plurality of flexible components includes a second central aperture, the elongate core being configured to extend through the second central aperture of each of the second plurality of flexible components, the first plurality of rigid components being stiffer than the second plurality of flexible components.

12. The method of forming a dynamic spinal stabilization rod of claim 11, further comprising:
d) threading the elongate core through the first central aperture of one of the first plurality of rigid components;
e) fixedly securing the one of the first plurality of rigid components to the elongate core using the locking mechanism;
f) threading the elongate core through the second central aperture of one of the second plurality of flexible components until the one of the second plurality of flexible components abuts the one of the first plurality of rigid components along the longitudinal extent of the elongate core; and
g) repeat steps d-f above for additional ones of the first plurality of rigid components and the second plurality of flexible components to form a central rod assembly including a series of rigid and flexible components.

13. The method of forming a dynamic spinal stabilization rod of claim 12, further comprising:
h) threading the elongate core through the first central aperture of a further additional one of the first plurality of rigid components, whereby a rigid component is disposed on opposed ends of the series of rigid and flexible components; and
i) trimming a length of the elongate core.

14. The method of forming a dynamic spinal stabilization rod of claim 12, further comprising:
h) mounting the central rod assembly to at least two bone anchors.

15. The method of forming a dynamic spinal stabilization rod of claim 14, wherein mounting the central rod assembly to the at least two bone anchors fixedly secures at least one rigid component to one of the two bone anchors.

16. The method of forming a dynamic spinal stabilization rod of claim 14, wherein mounting the central rod assembly to the at least two bone anchors fixedly secures at least two rigid components to one of the two bone anchors.

17. The method of forming a dynamic spinal stabilization rod of claim 11, wherein the first plurality of rigid components is greater in number than the second plurality of flexible components.
